SEQUENCE LISTING (54 sequences)

SEQ ID NO: 1-2 - skipped (2 entries)

SEQ ID NO: 3 - protein, 18 aa, synthetic construct
  REGION 1..18: note = Rigid linker 1
EAAAAKEAAAAKEAAAAK

SEQ ID NO: 4 - protein, 613 aa, Pseudomonas aeruginosa
  REGION 1..613: note = Full length Pseudomonas Exotoxin A (PE)
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 5 - protein, 26 aa, synthetic construct
  REGION 1..26: note = PE translocation peptide (PE280-305, minimal)
GWEQLEQCGYPVQRLVALYLAARLSW

SEQ ID NO: 6 - protein, 34 aa, synthetic construct
  REGION 1..34: note = PE translocation peptide (PE280-313)
GWEQLEQCGYPVQRLVALYLAARLSWNQVDQVIR

SEQ ID NO: 7 - protein, 46 aa, synthetic construct
  REGION 1..46: note = PE translocation peptide (PE268-313)
PLETFTRHRQPRGWEQLEQCGYPVQRLVALYLAARLSWNQVDQVIR

SEQ ID NO: 8 - protein, 61 aa, synthetic construct
  REGION 1..61: note = PE translocation peptide (PE253-313)
GGSLAALTAHQACHLPLETFTRHRQPRGWEQLEQCGYPVQRLVALYLAARLSWNQVDQVIR

SEQ ID NO: 9 - protein, 112 aa, synthetic construct
  REGION 1..112: note = PE translocation peptide (PE253-364)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAN

SEQ ID NO: 10 - protein, 293 aa, Shigella dysenteriae
  REGION 1..293: note = Full length Shiga toxin (Stx) subunit A
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 11 - protein, 293 aa, Escherichia coli
  REGION 1..293: note = Full length Shiga-like toxin I (Slt-I) subunit A
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 12 - protein, 8 aa, synthetic construct
  REGION 1..8: note = Stx translocation peptide (Stx240-247, minimal)
LNCHHHAS

SEQ ID NO: 13 - protein, 12 aa, synthetic construct
  REGION 1..12: note = Stx translocation peptide (Stx240-251)
LNCHHHASRVAR

SEQ ID NO: 14 - protein, 37 aa, synthetic construct
  REGION 1..37: note = Stx translocation peptide (Stx211-247)
PDYHGQDSVRVGRISFGSINAILGSVALILNCHHHAS

SEQ ID NO: 15 - protein, 41 aa, synthetic construct
  REGION 1..41: note = Stx translocation peptide (Stx211-251)
PDYHGQDSVRVGRISFGSINAILGSVALILNCHHHASRVAR

SEQ ID NO: 16 - protein, 84 aa, synthetic construct
  REGION 1..84: note = Stx translocation peptide (Stx168-251)
ALRFRQIQRGFRTTLDDLSGRSYVMTAEDVDLTLNWGRLSSVLPDYHGQDSVRVGRISFGSINAILGSVALILNCHHHASRVAR

SEQ ID NO: 17 - protein, 261 aa, Homo sapiens
  REGION 1..261: note = Full length CD40 ligand (CD40L1-261)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - protein, 215 aa, synthetic construct
  REGION 1..215: note = Truncated CD40 ligand (CD40L47-261)
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 19 - protein, 154 aa, synthetic construct
  REGION 1..154: note = Truncated CD40 ligand (CD40L108-261, also referred to as 18sCD40L)
ENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 246 aa, synthetic construct
  REGION 1..246: note = Anti-CD40 scFv (VH-L-VL)
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 21 - protein, 246 aa, synthetic construct
  REGION 1..246: note = Anti-CD40 scFv (VL-L-VH)
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGGGGGSGGGGSGGGGSEVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 22 - protein, 119 aa, synthetic construct
  REGION 1..119: note = VH of the anti-CD40 scFv
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 23 - protein, 112 aa, synthetic construct
  REGION 1..112: note = VL of the anti-CD40 scFv
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 24 - protein, 10 aa, synthetic construct
  REGION 1..10: note = VH CDR1
GFTFSTYGMH

SEQ ID NO: 25 - protein, 26 aa, synthetic construct
  REGION 1..26: note = VH CDR2
GKGLEWLSYISGGSSYIFYADSVRGR

SEQ ID NO: 26 - protein, 13 aa, synthetic construct
  REGION 1..13: note = VH CDR3
CARILRGGSGMDL

SEQ ID NO: 27 - protein, 15 aa, synthetic construct
  REGION 1..15: note = VL CDR1
CTGSSSNIGAGYNVY

SEQ ID NO: 28 - protein, 7 aa, synthetic construct
  REGION 1..7: note = VL CDR2
GNINRPS

SEQ ID NO: 29 - protein, 12 aa, synthetic construct
  REGION 1..12: note = VL CDR3
CAAWDKSISGLV

SEQ ID NO: 30 - protein, 4 aa, synthetic construct
  REGION 1..4: note = ER retention sequence
KDEL

SEQ ID NO: 31 - protein, 12 aa, synthetic construct
  REGION 1..12: note = ER retention sequence
KKDLRDELKDEL

SEQ ID NO: 32 - protein, 13 aa, synthetic construct
  REGION 1..13: note = ER retention sequence
KKDELRDELKDEL

SEQ ID NO: 33 - protein, 13 aa, synthetic construct
  REGION 1..13: note = ER retention sequence
KKDELRVELKDEL

SEQ ID NO: 34 - protein, 12 aa, synthetic construct
  REGION 1..12: note = ER retention sequence
KDELKDELKDEL

SEQ ID NO: 35 - protein, 28 aa, synthetic construct
  REGION 1..28: note = CD28 consensus sequence
  SITE 8: note = X is I or L
  SITE 10: note = X is V, F or A
  SITE 11: note = X is M or L
  SITE 17: note = X is L or I
TDIYFCKXEXXYPPPYXDNEKSNGTIIH

SEQ ID NO: 36 - protein, 28 aa, synthetic construct
  REGION 1..28: note = CD28-activating peptide (minimal)
TDIYFCKIEVMYPPPYLDNEKSNGTIIH

SEQ ID NO: 37 - protein, 53 aa, synthetic construct
  REGION 1..53: note = CD28-activating peptide
NCDGKLGNESVTFYLQNLYVNQTDIYFCKIEVMYPPPYLDNEKSNGTIIHVKG

SEQ ID NO: 38 - protein, 6 aa, synthetic construct
  REGION 1..6: note = Rigid linker
EAAAAK

SEQ ID NO: 39 - protein, 1273 aa, Severe acute respiratory syndrome coronavirus 2 (SARS-CoV-2)
  REGION 1..1273: note = SARS-CoV2 spike (S) protein (NCBI accession number: YP_009724390.1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - protein, 672 aa, Severe acute respiratory syndrome coronavirus 2 (SARS-CoV-2)
  REGION 1..672: note = S1 subunit of S protein (Corresponding to 14-685 residues of SEQ ID NO: 39)
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 41 - protein, 223 aa, Severe acute respiratory syndrome coronavirus 2 (SARS-CoV-2)
  REGION 1..223: note = Receptor-binding domain of S protein (SRBD) (Corresponding to 319-541 residues of SEQ ID NO: 39)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 42 - protein, 367 aa, synthetic construct
  REGION 1..367: note = Truncated S protein (S319-685) (Corresponding to 319-685 residues of SEQ ID No. 39)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 43 - protein, 588 aa, Severe acute respiratory syndrome coronavirus 2 (SARS-CoV-2)
  REGION 1..588: note = S2 subunit of S protein (Corresponding to 686-1273 residues of SEQ ID NO: 39)
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 75 aa, Severe acute respiratory syndrome coronavirus 2 (SARS-CoV-2)
  REGION 1..75: note = SARS-CoV2 envelope (E) protein (NCBI accession number: YP_009724392.1)
MYSFVSEETGTLIVNSVLLFLAFVVFLLVTLAILTALRLCAYCCNIVNVSLVKPSFYVYSRVKNLNSSRVPDLLV

SEQ ID NO: 45 - protein, 222 aa, Severe acute respiratory syndrome coronavirus 2 (SARS-CoV-2)
  REGION 1..222: note = SARS-CoV2 membrane (M) protein (NCBI accession number: YP_009724393.1)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 46 - protein, 419 aa, Severe acute respiratory syndrome coronavirus 2 (SARS-CoV-2)
  REGION 1..419: note = SARS-CoV2 nucleocapsid (N) protein (NCBI accession number: YP_009724397.2)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 47 - protein, 462 aa, synthetic construct
  REGION 1..462: note = Fusion protein CD40L-TPE-SRBD
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFTRAKDEL

SEQ ID NO: 48 - protein, 606 aa, synthetic construct
  REGION 1..606: note = Fusion protein CD40L-TPE-S319-685
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARTRAKDEL

SEQ ID NO: 49 - protein, 536 aa, synthetic construct
  REGION 1..536: note = Fusion protein CD40L-TPE-E/M
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMYSFVSEETGTLIVNSVLLFLAFVVFLLVTLAILTALRLCAYCCNIVNVSLVKPSFYVYSRVKNLNSSRVPDLLVMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQTRAKDEL

SEQ ID NO: 50 - protein, 658 aa, synthetic construct
  REGION 1..658: note = Fusion protein CD40L-TPE-N
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQATRAKDEL

SEQ ID NO: 51 - protein, 469 aa, synthetic construct
  REGION 1..469: note = Fusion protein SRBD-TStx-CD40L
MKLTMGSSHHHHHHSSGLVPRGSHRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 52 - protein, 613 aa, synthetic construct
  REGION 1..613: note = Fusion protein S319-685-TStx-CD40L
MKLTMGSSHHHHHHSSGLVPRGSHRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 53 - protein, 543 aa, synthetic construct
  REGION 1..543: note = Fusion protein E/M-TStx-CD40L
MKLTMGSSHHHHHHSSGLVPRGSHMYSFVSEETGTLIVNSVLLFLAFVVFLLVTLAILTALRLCAYCCNIVNVSLVKPSFYVYSRVKNLNSSRVPDLLVMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 54 - protein, 665 aa, synthetic construct
  REGION 1..665: note = Fusion protein N-TStx-CD40L
MKLTMGSSHHHHHHSSGLVPRGSHMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQATRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2024/0398936 A1
(43) Pub. Date: Dec. 5, 2024

(54) IMMUNOGENIC FUSION PROTEINS AGAINST CORONAVIRUS

(71) Applicant: NAVICURE BIOPHARMACEUTICALS LIMITED, Apia City (WS)

(72) Inventor: Chia-Mao WU, Hsinchu City (TW)

(21) Appl. No.: 18/698,008

(22) PCT Filed: Oct. 18, 2022
(86) PCT No.: PCT/US2022/078297

§ 371 Date: Apr. 2, 2024

Related U.S. Application Data
(60) Provisional application No. 63/271,982, filed on Oct. 26, 2021.
(60) Provisional application No. 63/271,987, filed on Oct. 26, 2021.

(51) Int. Cl. A61K 39/215; A61K 38/19; A61P 37/04; C07K 14/005; C07K 14/52

(52) U.S. Cl. CPC A61K 39/215; A61K 38/19; A61P 37/04; C07K 14/005; C07K 14/52; C07K 2319/01; C07K 2319/55; C12N 2770/20022; C12N 2770/20034; C12N 2770/20071

(57) ABSTRACT

Immunogenic fusion proteins against coronavirus. A fusion protein is disclosed, which comprises a CD40-binding domain; an antigen of SARS-CoV2; a translocation domain located between the CD40-binding domain and the antigen, and a furin and/or cathepsin L cleavage site located between the CD40-binding domain and the translocation domain. In one embodiment, the translocation domain is a Shiga toxin (Stx) translocation peptide, and the antigen is located at the N-terminal of the fusion protein. In another embodiment, the translocation domain is a Pseudomonas Exotoxin A (PE) translocation peptide, and the CD40-binding domain is located at the N-terminal of the fusion protein. Also disclosed are pharmaceutical compositions, expression vectors and use of the fusion proteins of the invention for eliciting an antigen-specific cell-mediated immune response, and/or for reducing, inhibiting, treating and/or ameliorating symptoms caused by SARS-CoV2 infection in a subject in need thereof.

### REFERENCE TO AN ELECTRONIC SEQUENCE LISTING

The contents of the electronic sequence listing (10040-003PCT_sequence listing_ST26.xml; size 79 KB; and creation date Oct. 4, 2022) is herein incorporated by reference in its entirety.

### FIELD OF THE INVENTION

The present invention relates generally to fusion proteins, and more specifically to immunogenic fusion proteins for eliciting antigen-specific cell-mediated immune responses against SARS-CoV2.

### BACKGROUND OF THE INVENTION

Severe Acute Respiratory Syndrome Coronavirus 2 (SARS-CoV2), a coronavirus causing global pandemic, was first identified in Wuhan, China in December 2019. SARS-CoV2 has infected hundreds of millions of people causing millions of deaths worldwide. SARS-CoV2's structural proteins are spike (S), envelope (E), membrane (M) and nucleocapsid (N) proteins. Spike protein S1 and S2 subunits are frequently used as a main target for vaccine development.

Several companies have developed vaccines against SARS-CoV2, including Pfizer/BioNTech (COMIRNATY®; a mRNA vaccine), Moderna (SPIKEVAX®; a mRNA vaccine), AstraZeneca (VAXZEVRIA®; an adenovirus-vector vaccine), Janssen (Ad26.COV2.S; an adenovirus vector-based vaccine), Sinovac (CoronaVac; an inactivated virus vaccine), Sinopharm (BBIBP-CorV; an inactivated virus vaccine) and Novavax (NVX-CoV2373; a subunit vaccine). All these vaccines are designed to target the spike protein (Franz X. Heinz et al. (2021), npj Vaccines, 6: 104). However, there is still a need to develop a more potent vaccine for combating the pandemic.

### SUMMARY OF THE INVENTION

In one aspect, the invention relates to a fusion protein comprising: (a) a CD40-binding domain; (b) an antigen of severe acute respiratory syndrome coronavirus 2 (SARS-CoV2); (c) a translocation domain located between the CD40-binding domain and the antigen; and (d) a furin and/or cathepsin L cleavage site located between the CD40-binding domain and the translocation domain.

In another aspect, the invention relates to a DNA fragment and an expression vector comprising the DNA fragment encoding a fusion protein of the invention. The invention further relates to a pharmaceutical composition or a vaccine composition comprising the fusion protein of the invention and a pharmaceutical acceptable carrier and/or an adjuvant.

The pharmaceutical or vaccine composition may further comprise an immune checkpoint antibody. The immune checkpoint antibody is capable of activating a T cell.

The invention further relates to a combination or a pharmaceutical composition comprising: (a) an immune checkpoint antibody; and (b) a fusion protein of the invention.

Yet in another aspect, the invention relates to use of a fusion protein, a combination, or a pharmaceutical composition in the manufacture of a medicament for eliciting an antigen-specific cell-mediated immune response against SARS-CoV2 infection, or for reducing, inhibiting, treating and/or ameliorating symptoms caused by SARS-CoV2 infection in a subject in need thereof. The subject may be a human.

The invention also relates to a fusion protein, a combination, a pharmaceutical composition or a vaccine composition for use in eliciting an antigen-specific cell-mediated immune response against SARS-CoV2 infection, or for use in reducing, inhibiting, treating and/or ameliorating symptoms caused by SARS-CoV2 infection in a subject in need thereof.

Alternatively, the invention relates to a method for eliciting an antigen-specific cell-mediated immune response against SARS-CoV2 infection, or for reducing, inhibiting, treating and/or ameliorating symptoms caused by SARS-CoV2 infection in a subject in need thereof, said method comprising administering a therapeutically effective amount of a fusion protein, a combination, a pharmaceutical composition or a vaccine according to the invention to the subject in need thereof.

### BRIEF DESCRIPTION OF DRAWINGS

FIGS. 1A-D and 2A-D are vector maps.

FIGS. 3A-F are schematic drawings illustrating various embodiments of the invention.

FIG. 4 shows an immunization scheme (upper panel), animal groups and respective dosing schedules (lower panel).

FIG. 5 is a graph showing IFN-γ inductions in CD4⁺ memory T cells in each animal group.

FIG. 6 is a graph showing IL-2 inductions in CD4⁺ memory T cells in each animal group.

FIG. 7 is a graph showing IFN-γ⁺ immunospot results of the splenocytes from each animal group.

FIG. 8 is a graph showing SARS-CoV2 antigen-specific antibody levels in each animal group.

FIG. 9 shows the lung viral titers of SARS-CoV2-infected animals prophylactically given a fusion protein of the invention.

FIG. 10 shows the lung viral titers of SARS-CoV2-infected animals treated with remdesivir or a fusion protein of the invention.

FIG. 11 shows the survival rate of SARS-CoV2-infected animals treated with remdesivir or a fusion protein of the invention.

### DETAILED DESCRIPTION OF THE INVENTION

### Definitions

Professional APCs and non-professional APCs use an MHC class I molecule to display endogenous peptides on the cell membrane. These peptides originate within the cell itself, in contrast to the exogenous antigen displayed by professional APCs using MHC class II molecules. Cytotoxic CD8+ T cells can interact with antigens presented by the MHC class I molecule.

CD40 is a costimulatory protein expressed on antigen-presenting cells (e.g., dendritic cells, macrophage and B cells). The binding of CD40L to CD40 activates antigen-presenting cells and induces a variety of downstream effects. CD40 is a drug target for cancer immunotherapy.

The term “a CD40-binding domain” refers to a protein that can recognize and binds to CD40. A CD40-binding domain may be selected from one of the following: CD40 ligand (CD40L) or a functional fragment thereof, an anti-CD40 antibody or a functional fragment thereof.

The terms “CD40L”, “CD40 ligand” and “CD154” are interchangeable. CD40L binds to CD40 (protein) on antigen-presenting cells (APC), which leads to many effects depending on the target cell type. CD40L plays a central role in co-stimulation and regulation of the immune response via T cell priming and activation of CD40-expressing immune cells. U.S. Pat. No. 5,962,406 discloses the nucleotide and amino acid sequence of CD40L.

The terms “anti-CD40 antibody”, “CD40-specific antibody”, “an antibody specifically against CD40” are interchangeable.

When the term “consist substantially of” is used in describing an amino acid sequence of a polypeptide, it means that the polypeptide may or may not have a starting amino acid “M” (translated from a start codon AUG) at N-terminal as a part of the polypeptide, depending on protein translation requirements. For example, when the second antigen fused to the first antigen, the starting amino acid “M” of the second antigen could be omitted or kept.

As used herein, “a translocation domain” is a polypeptide having biological activity in translocating a fused or linked antigen across an endosomal membrane into cytosol of a cell. The translocation domain guides or facilitates the antigen toward class I major histocompatibility complex (MHC-1) pathway (i.e., a cytotoxic T cell pathway) for antigen presentation.

The term “a Pseudomonas Exotoxin A (PE) translocation peptide (T^{PE})” refers to a PE domain II peptide or a functional fragment thereof that has the biological activity in translocation.

The term “a Shiga toxin (Stx) translocation peptide (T^{Stx})” refers to a Stx translocating domain or a functional fragment thereof that has the biological activity in translocation.

The terms “furin and/or cathepsin L” or “furin/cathepsin L” are interchangeable.

The term “furin and/or cathepsin L cleavage site” refers to a short peptide sequence having at least four amino acids that can be cleaved by furin or cathepsin L, or by both furin and cathepsin L. Said cleavage site is a furin and/or cathepsin L protease sensitive site. It may be a peptide linker comprising said cleavage site that is introduced into the fusion protein. In addition, the furin and/or cathepsin L cleavage site may be a PE or Stx intrinsic protease cleavage site present in or adjacent to the translocation domain of the fusion protein.

The terms “antigen” and “immunogen” are interchangeable. An antigen refers to an antigenic protein or polypeptide derived from SARS-CoV2. Said antigen comprises at least one epitope for inducing desirable immune response. In some embodiments, the antigen is a polypeptide of at least 8 amino acids in length derived from spike (S) protein, envelope (E) protein, membrane (M) protein or nucleocapsid (N) protein of SARS-CoV2.

The terms “an antigen of SARS-CoV2”, “a SARS-CoV2 antigen” and “a SARS-CoV2 viral protein antigen” are interchangeable.

The terms “selected” and “derived” are interchangeable. Thus, the terms “a polypeptide selected from a protein”, “a polypeptide selected from a region of a protein” and “a polypeptide derived from a protein” are interchangeable.

Cluster of Differentiation 28 (CD28) is a T-cell-specific surface glycoprotein. A CD28 receptor is stimulated during the contact of T cells with antigen-presenting cells. Its function is involved in T-cell activation, the induction of cell proliferation and cytokine production and promotion of T-cell survival.

The term “an effective amount” or “a therapeutically effective amount” refers to the amount of an active fusion protein that is required to confer a therapeutic effect on the treated subject. Effective doses will vary, as recognized by those skilled in the art, depending on rout of administration, excipient usage, and the possibility of co-usage with other therapeutic treatment.

The term “treating”, or “treatment” refers to administration of an effective amount of the fusion protein to a subject in need thereof, who has cancer or infection, or a symptom or predisposition toward such a disease, with the purpose to cure, alleviate, relieve, remedy, ameliorate, or reduce, inhibit the disease, the symptoms of it, or the predisposition towards it. Such a subject can be identified by a health care professional based on results from any suitable diagnostic method.

By “0 to 12 repeats” or “2 to 6 repeats”, it means that all integer unit amounts within the range “0 to 12” or “2 to 6” are specifically disclosed as part of the invention. Thus, 0, 1, 2, 3, 4, . . . 10, 11 and 12” or “2, 3, 4, 5 and 6” unit amounts are included as embodiments of this invention.

Abbreviations: MCS, multiple cloning sites; a.a., amino acid; anti-PD-1, anti-programmed cell death-1; anti-PD-L1, anti-programmed cell death ligand-1; Rap1, Ras-proximate-1 or Ras-related protein 1; CD40, Cluster of differentiation 40; CDR, Complementarity-determining region; SARS-CoV2, severe acute respiratory syndrome coronavirus 2.

### Fusion Proteins

In one aspect, the invention relates to a fusion protein comprising: (a) a CD40-binding domain; (b) an antigen of SARS-CoV2; (c) a translocation domain, located between the CD40-binding domain and the antigen; and (d) a furin and/or cathepsin L cleavage site, located between the CD40-binding domain and the translocation domain.

The fusion proteins of the invention can elicit an antigen-specific T cell immune response via MHC class I antigen presentation pathway. They share a common mechanism of action. Using CD40L-T^{PE}-Ag (Ag stands for any suitable antigen) as an example, the mechanism of action is as follows:
(1) CD40L-T^{PE}-Ag binds to a CD40-expressing cell (e.g., dendritic cell or macrophage) and is internalized via a CD40-mediated endocytosis;
(2) CD40L-T^{PE}-Ag is cleaved by furin protease and/or cathepsin L protease within an endosome so as to remove the CD40L fragment away from the T^{PE}-Ag fragment;
(3) the T^{PE}-Ag fragment is translocated across the endosomal membrane and enter the cytosol;
(4) the T^{PE}-Ag fragment is digested by cytosol proteasomes to generate small antigens comprising epitopes;
(5) the small antigens are delivered via MHC class I pathway for antigen presentation; and
(6) a CD8⁺ T cell specific immune response is induced or enhanced by T-cell recognizing these presented antigens.

The same mechanism of action applies to Ag-T^{Stx}-CD40L, in which furin and/or cathepsin L protease cleavage would remove the Ag-T^{Stx} fragment away from the CD40L fragment. Thus, the Ag-T^{Stx} fragment would be translocated across the endosomal membrane, enter the cytosol, digested by cytosol proteasomes to generate small antigens comprising epitopes. The small antigens would be delivered via MHC class I pathway for antigen presentation and a CD8⁺ T cell-specific immune response would be induced or enhanced by T-cell recognizing the presented antigens.

No furin and/or cathepsin L cleavage site is present between the antigen and the translocation domain in the fusion protein of the invention. The presence of a furin and/or cathepsin L cleavage site and its location in the fusion protein permits removal of CD40-binding domain from the fusion protein after the furin and/or cathepsin L cleavage.

In one embodiment, the furin and/or cathepsin L cleavage site comprises or consists of 4-20 amino acids, preferred 4-10 amino acids, and more preferred 4-6 amino acids. In another embodiment, a furin and/or cathepsin L cleavage site comprises, consists of, or is SEQ ID NO: 1 or 2.

In another embodiment, the fusion protein of the invention further comprises a peptide linker between the CD40-binding domain and the translocation domain, wherein the furin and/or cathepsin L cleavage site is present in said peptide linker. The peptide linker may comprise (a) a rigid linker (EAAAAK)ₙ or (SEQ ID NO: 38); and (b) a cleavable linker comprising the furin and/or cathepsin L cleavage site, wherein n is an integer from 0-12, preferably from 2-6, more preferably from 3-4, and said furin and/or cathepsin L cleavage site comprises SEQ ID NO: 1 or 2. In one embodiment, the peptide linker comprises (EAAAAK)₃ and RX¹RX²X³R (SEQ ID NO: 2; wherein X¹ is A, X² is Y, X³ is K). In another embodiment, the peptide linker comprises RX¹X²R (SEQ ID NO: 1; wherein X¹ is V, X² is A) and (EAAAAK)₃.

The translocation domain and the antigen are located within the fusion protein in such an orientation and/or relation that permits the translocation domain to translocate the antigen across the membrane of the endosome and enter the cytosol and facilitate the antigen toward MHC class I pathway for antigen presentation in said CD40-expressing cell.

The translocation domain may be selected from Pseudomonas Exotoxin A (PE) or Shiga toxin (Stx). In one embodiment, the translocation domain comprises or is a Pseudomonas Exotoxin A (PE) translocation peptide (T^{PE}), with the proviso that the CD40-binding domain is located at the N-terminal of the fusion protein. In another embodiment, the translocation domain comprises or is a Shiga toxin (Stx) translocation peptide (T^{Stx}), with the proviso that the antigen is located at the N-terminal of the fusion protein.

In one embodiment, a fusion protein of the invention sequentially (from N- to C-terminal) comprises: (a) a CD40-binding domain; (b) a furin and/or cathepsin L cleavage site; (c) a translocation domain comprising a PE translocation peptide (T^{PE}); and (d) an antigen of SARS-CoV2. In another embodiment, a fusion protein of the invention sequentially (from N- to C-terminal) comprises: (a) a CD40-binding domain; (b) a peptide linker comprising a furin and/or cathepsin L cleavage site; (c) a translocation domain comprising a PE translocation peptide (T^{PE}); and (d) an antigen of SARS-CoV2. In another embodiment, a fusion protein of the invention sequentially (from N-terminal to C-terminal) comprises: (a) an antigen of SARS-CoV2; (b) a translocation domain comprising a Stx translocation peptide (T^{Stx}); (c) a furin and/or cathepsin L cleavage site; and (d) a CD40-binding domain. In another embodiment, a fusion protein of the invention sequentially (from N-terminal to C-terminal) comprises: (a) an antigen of SARS-CoV2; (b) a translocation domain comprising a Stx translocation peptide (T^{Stx}); (c) a peptide linker comprising a furin and/or cathepsin L cleavage site; and (d) a CD40-binding domain.

The T^{PE} or T^{Stx} is a functional moiety having a biological activity in translocation. The furin and/or cathepsin L cleavage site may be one selected from SEQ ID NO: 1, SEQ ID NO: 2 or an intrinsic furin cleavage site within or derived from PE or Stx. In one embodiment, a PE translocation peptide (T^{PE}) is domain II (a.a. residues 253-364; SEQ ID NO: 9) of Pseudomonas Exotoxin A protein (full-length PE, SEQ ID NO: 4) or a functional moiety thereof. In another embodiment, the PE translocation peptide (T^{PE}) consists of 26-112 a.a. residues in length. The PE translocation peptide (T^{PE}) comprises a minimal functional fragment of GWEQLEQCGYPVQRLVALYLAARLSW (SEQ ID NO: 5). In one embodiment, a PE translocation peptide (T^{PE}) comprises an amino acid sequence that is at least 95%, 97% or 99% identical to SEQ ID NO: 5, 6, 7, 8 or 9. In another embodiment, a T^{PE} comprises an amino acid sequence selected from the group consisting of SEQ ID NO: 5, 6, 7, 8 and 9. In another embodiment, a T^{PE} is PE₂₈₀₋₃₀₅ (SEQ ID NO: 5), PE₂₈₀₋₃₁₃ (SEQ ID NO: NO: 6), PE₂₆₈₋₃₁₃ (SEQ ID NO: NO: 7), PE₂₅₃₋₃₁₃ (SEQ ID NO: 8), or PE₂₅₃₋₃₆₄ (SEQ ID NO: 9; full-length PE domain II).

In one embodiment, a Stx translocation peptide (T^{Stx}) is a functional fragment of Shiga toxin (Stx) subunit A (SEQ ID NO: 10) or Shiga-like toxin I (Slt-I) subunit A (SEQ ID NO: 11). A Stx translocation peptide has translocation function but has no cytotoxic effect of subunit A. Sequence identify between Shiga toxin (Stx) subunit A and Slt-I subunit A is 99% and the two proteins has only one amino acid difference. In another embodiment, the Stx translocation peptide (T^{lx}) consists of 8-84 a.a. residues in length. The Stx translocation peptide (T^{Stx}) comprises a minimal functional fragment of LNCHHHAS (SEQ ID NO: 12). In one embodiment, a Stx translocation peptide (T^{lx}) comprises an amino acid sequence that is at least 95%, 97% or 99% identical to SEQ ID NO: 12, 13, 14, 15 or 16. In another embodiment, a T^{Stx} comprises an amino acid sequence selected from the group consisting of SEQ ID NOs: 12, 13, 14, 15 and 16. In another embodiment, a T^{Stx} is Stx₂₄₀₋₂₄₇ (SEQ ID NO: 12), Stx₂₄₀₋₂₅₁ (SEQ ID NO: 13), Stx₂₁₁₋₂₄₇ (SEQ ID NO: 14), Stx₂₁₁₋₂₅₁ (SEQ ID NO: 15) or Stx₁₆₈₋₂₅₁ (SEQ ID NO: 16) of Stx subunit A.

A CD40-binding domain permits a fusion protein of the invention to bind to a CD40 receptor on a CD40-expressing cell (e.g., dendritic cell or macrophage). A CD40-binding domain may be one selected from the group consisting of (i) a CD40 ligand (CD40L) or a functional fragment thereof; and (ii) a CD40-specific antibody or a functional fragment thereof. In one embodiment, a functional fragment of CD40L is a truncated CD40L substantially lacking the transmembrane and cytoplasmic regions of full-length CD40L₁₋₂₆₁ protein (SEQ ID NO: 17).

In another embodiment, a CD40L or a functional fragment thereof consists of 154-261 a.a. residues in length. In another embodiment, a CD40L comprises a minimal functional fragment of SEQ ID NO: 19. Further in another embodiment, a CD40L or a functional fragment thereof consists of 154-261 a.a. residues in length and said CD40L comprises a minimal functional fragment of SEQ ID NO: 19. In one embodiment, a CD40L comprises an amino acid sequence that is at least 95%, 97% or 99% identical to SEQ ID NO: 17, 18 or 19. In another embodiment, a CD40L is selected from the group consisting of CD40L₁₋₂₆₁ (SEQ ID NO: 17), CD40L₄₇₋₂₆₁ (SEQ ID NO: 18) and CD40L₁₀₈₋₂₆₁ (SEQ ID NO: 19). In another embodiment, a CD40-binding domain is a CD40-specific antibody (or anti-CD40 antibody). A CD40-specific antibody is an antibody specifically recognizing and binding to CD 40 protein. A CD40-specific antibody can bind to CD40 protein on a CD40-expressing cell.

In one embodiment, the CD40-specific antibody comprises a heavy chain variable domain (V_{H}) and a light chain variable domain (V_{L}), wherein the V_{H} comprises the amino acid sequence of SEQ ID NO: 22; and the V_{L} comprises the amino acid sequence of SEQ ID NO: 23. In another embodiment, the CD40-specific antibody is selected from the group consisting of a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab₂, a minibody and a fully antibody. In another embodiment, the CD40-binding domain is a CD40-specific scFv (anti-CD40 scFv) comprising a heavy chain variable domain (V_{H}), a light chain variable domain (V_{L}) and a flexible linker (L) connecting the V_{H} and the V_{L}. In one embodiment, the CD40-specific scFv comprises SEQ ID NO: 20 or 21.

In another embodiment, the CD40-binding domain is (i) a CD40-specific antibody or a binding fragment thereof, or (ii) a CD40-specific single chain variable fragment (scFv) or a binding fragment thereof; said CD40-specific antibody or said CD40-specific scFv comprising a V_{H} and a V_{L}, wherein: (a) the V_{H} comprises SEQ ID NO: 22; and (b) the V_{L} comprises SEQ ID NO: 23.

In another embodiment, the CD40-specific antibody or CD40-specific scFv comprises a V_{H} and a V_{L}, the V_{H} comprising V_{H} CDR1, V_{H} CDR2 and V_{H} CDR3; and the V_{L} comprising V_{L} CDR1, V_{L} CDR2 and V_{L} CDR3, wherein: (i) the V_{H} CDR1, V_{H} CDR2 and V_{H} CDR3 comprises SEQ ID NO: 24, 25 and 26, respectively; and (ii) the V_{L} CDR1, V_{L} CDR2 and V_{L} CDR3 comprises SEQ ID NO: 27, 28 and 29, respectively.

In another embodiment, the CD40-binding domain is a CD40-specific scFv comprising a V_{H} and a V_{L}, wherein: (a) the V_{H} comprises SEQ ID NO: 22; and (b) the V_{L} comprises SEQ ID NO: 23.

In another embodiment, a fusion protein of the invention further comprises an endoplasmic reticulum (ER) retention sequence located at the C-terminal of the antigen, with the proviso that the translocation domain comprises a PE translocation peptide (T^{PE}). The ER retention sequence may comprise SEQ ID NO: 30, 31, 32, 33 or 34. In one embodiment, the ER retention sequence is SEQ ID NO: 30.

In another embodiment, the fusion protein of the invention further comprises a CD28-activating peptide located between the CD40-binding domain and the furin and/or cathepsin L cleavage site.

In some embodiments, the CD28-activating peptide consists of 28-53 a.a. residues in length. In some embodiments, the CD28-activating peptide comprises a minimal functional fragment of SEQ ID NO: 35. In a particular embodiment, the CD28-activating peptide consists of 28-53 a.a. residues in length and said CD28-activating peptide comprises a minimal functional fragment of SEQ ID NO: 35.

In another embodiment, the CD28-activating peptide comprises an amino acid sequence that is at least 95%, 97% or 99% identical to SEQ ID NO: 35, 36 or 37. In another embodiment, the CD28-activating peptide comprises an amino acid sequence selected from the group consisting of SEQ ID NO: 35, 36 and 37. In another embodiment, the CD28-activating peptide is SEQ ID NO: 35, 36 or 37.

In one embodiment, SARS-CoV2 is a Wuhan-Hu-1 strain with a NCBI reference number NC_045512.2, or a variant thereof.

In another embodiment, SARS-CoV2 is a viral variant comprising at least one amino acid mutation as follows:
(a) the at least one amino acid mutation in S protein selected from the group consisting of L5F, S12F, S13I, L18F, T19R, T20N, P26S, Q52R, A67V, DEL69/70, V70F, G75V, T76I, D80G, D80A, T95I, D138Y, DEL141/143, G142D, DEL144/145, Y144S, Y145N, W152R, W152C, E154Q, E154K, DEL157/158, F157S, R190S, D215G, A222V, DEL241/243, DEL243/244, DEL247/253, D253G, W258L, Y265C, R346K, R346S, K417N, K417T, N439K, L452R, L452Q, S477N, T478K, E484K, E484Q, F490S, N501Y, K558N, A570D, D614G, H655Y, Q677H, P681R, P681H, A688V, A701V, T716L, D796H, S813N, T859N, F888L, A899S, D950N, D950H, Q957R, S982A, T1027I, Q1071H, E1072K, E1092K, H1101Y, H1101D, D1118H, I1130V, D1139H and V1176F, wherein a reference sequence for the mutation is SEQ ID NO: 39 of the S protein;
(b) the at least one amino acid mutation in E protein selected from the group consisting of L21F, S68F and P71L, wherein a reference sequence for the mutation is SEQ ID NO: 44 of the E protein;
(c) the at least one amino acid mutation in the M protein selected from the group consisting of L29F, A63T, I82T, I82S and H125Y, wherein a reference sequence for the mutation is SEQ ID NO: 45 of the M protein; and
(d) the at least one amino acid mutation in N protein selected from the group consisting of D3L, DEL3, D3Y, A12G, P13L, D63G, P67S, P80R, A119S, I157V, P199L, S202R, R203M, R203K, G204R, G204P, T205I, DEL209, G212V, G214C, G215C, M234I, S235F, K256R, T366I and D377Y, wherein a reference sequence for the mutation is SEQ ID NO: 46 of the N protein.

In each mutation mentioned above, the number represents the corresponding amino acid (a.a.) position in the protein sequence. The capital letter adjacent to the number is Single-Letter Amino Acid Code. The capital letters “DEL” adjacent to the number represent a.a. deletion in the corresponding position. Thus, mutation “D3L” means that a.a. at position 3 of the indicated protein sequence is mutated from D (aspartic acid) to L (Leucine). Mutation “DEL3” means that a.a. at position 3 of the indicated protein sequence is deleted.

According to the WHO label, SARS-CoV2 viral variants include, but not limited to, (1) variants of concern, such as Alpha (B.1.1.7), Beta (B.1.351), Gamma (P.1), Delta (B.1.617.2), and Omicron (BA.1, BA.2, BA.5 or other lineages) variants, and (2) variants of interest, such as Eta, Iota, Kappa, Lambda and Mu variants. Each variant comprises at least one amino acid mutation as compared to the Wuhan-Hu-1 strain (NCBI reference number NC_045512.2). In one embodiment, said variant at least comprises mutation D614G in the spike protein. Fusion proteins of the invention make it feasible to utilize suitable antigens or conserved regions selected from SARS-CoV2 or variants thereof to make vaccines for inducing potent adaptive immune responses across variants.

An antigen in the fusion protein of the invention may comprise a polypeptide selected from spike (S) protein, envelope (E) protein, membrane (M) protein or nucleocapsid (N) protein of SARS-CoV2 (including the Wuhan-Hu-1 strain or any viral variant thereof).

In one embodiment, the antigen comprises at least one polypeptide selected from the group consisting of:
(a) a polypeptide of 8-1273 a.a. residues selected from a region of SEQ ID NO: 39 of S protein, wherein the polypeptide optionally comprises at least one amino acid mutation selected from the group consisting of L5F, S12F, S13I, L18F, T19R, T20N, P26S, Q52R, A67V, DEL69/70, V70F, G75V, T76I, D80G, D80A, T95I, D138Y, DEL141/143, G142D, DEL144/145, Y144S, Y145N, W152R, W152C, E154Q, E154K, DEL157/158, F157S, R190S, D215G, A222V, DEL241/243, DEL243/244, DEL247/253, D253G, W258L, Y265C, R346K, R346S, K417N, K417T, N439K, L452R, L452Q, S477N, T478K, E484K, E484Q, F490S, N501Y, K558N, A570D, D614G, H655Y, Q677H, P681R, P681H, A688V, A701V, T716L, D796H, S813N, T859N, F888L, A899S, D950N, D950H, Q957R, S982A, T1027I, Q1071H, E1072K, E1092K, H1101Y, H1101D, D1118H, I1130V, D1139H and V1176F;
(b) a polypeptide of 8-75 a.a. residues selected from a region of SEQ ID NO: 44 of E protein, wherein the polypeptide optionally comprises at least one amino acid mutation selected from the group consisting of L21F, S68F and P71L;
(c) a polypeptide of 8-222 a.a. residues selected from a region of SEQ ID NO: 45 of M protein, wherein the polypeptide optionally comprises at least one amino acid mutation selected from the group consisting of L29F, A63T, I82T, I82S and H125Y; and
(d) a polypeptide of 8-491 a.a. residues selected from a region of SEQ ID NO: 46 of N protein, wherein the polypeptide optionally comprises at least one amino acid mutation selected from the group consisting of D3L, DEL3, D3Y, A12G, P13L, D63G, P67S, P80R, A119S, I157V, P199L, S202R, R203M, R203K, G204R, G204P, T205I, DEL209, G212V, G214C, G215C, M234I, S235F, K256R, T366I and D377Y.

In one embodiment, the antigen is a truncated S protein of 8-1273 a.a. residues, preferably 100-800 a.a. residues, and more preferably 200-700 a.a. residues. The truncated S protein may be selected from SEQ ID NO: 40, 41, 42 or 43.

The antigen may be a fusion antigen comprising at least two polypeptides independently derived from two different proteins chosen from S, E, M and N proteins of SARS-CoV2. In one embodiment, the antigen is a fusion antigen comprising at least two polypeptides independently derived from S and M proteins, or derived from S and N proteins, or derived from E and M proteins of SARS-CoV2. For example, the antigen in the fusion protein CD40L-T^{PE}-E/M (SEQ ID No: 49) is a fusion antigen which comprises two polypeptides independently derived from E and M proteins of SARS-CoV2.

In one embodiment, the antigen comprises an amino acid sequence that is at least 80%, 85%, 90%, 95% or 99% identical to SEQ ID No: 39, 40, 41, 42, 43, 44, 45 or 46. In another embodiment, the antigen is an amino acid sequence that is at least 80%, 85%, 90%, 95% or 99% identical to SEQ ID No: 39, 40, 41, 42, 43, 44, 45 or 46. In another embodiment, the antigen is SEQ ID No: 39, 40, 41, 42, 43, 44, 45 or 46. Said antigen comprises at least one epitope for inducing a desired immune response, preferably containing 1 to 50 epitopes, more preferably containing 1 to 20 epitopes.

For developing SARS-CoV2 peptide vaccines, numerous epitope candidates have been designed in silico and studied to understand their activities of inducing B cell and/or T cell immune response (e.g., U.S. Ser. No. 10/973,908 B1; U.S. Ser. No. 10/973,909 B1; Christof C. Smith et al., (2021), Genome Medicine, 13: 101). These epitopes could be applied as parts of the antigen used in the fusion protein of the invention.

The antigen-carrying fusion proteins of the invention are expected to elicit potent immune responses, rather than simply and directly using these antigens/epitopes. Examples of suitable antigens/epitopes may be selected from corresponding positions in each reference sequence of SARS-CoV2 protein below (e.g., S₄₈₈₋₅₂₅ means an amino acid sequence corresponding to positions 488-525 of said S protein sequence):
(a) S₄₈₈₋₅₂₅, S₄₅₀₋₄₆₉, S₂₃₁₋₂₄₅, S₂₆₅₋₂₇₉, S₈₆₅₋₈₇₉, S₁₀₁₄₋₁₀₂₈, S₄₄₆₋₄₆₀, S₄₄₈₋₄₆₂, S₄₄₉₋₄₆₃, S₄₅₁₋₄₆₃, S₄₅₃₋₄₆₇, S₄₅₄₋₄₆₈, S₄₅₆₋₄₇₀, S₄₅₈₋₄₇₂, S₄₆₁₋₄₇₅, S₄₆₈₋₄₈₂, S₅₇₀₋₅₈₄, S₅₇₉₋₅₉₃, S₇₉₉-S₈₁₃, S₈₀₀₋₈₁₄, S₈₄₋₁₀₄, S₂₂₅₋₂₄₅, S₂₆₁₋₂₈₁, S₈₆₅₋₈₈₅, S₉₆₆₋₉₈₆, S₁₀₀₈₋₁₀₂₈, S₄₄₈₋₄₆₈, S₄₅₃₋₄₇₃, S₄₆₂₋₄₈₂, S₅₆₄₋₅₈₄, S₅₇₉₋₅₉₉, S₅₈₀₋₆₀₀, S₇₉₉₋₈₁₉, S₈₀₉₋₈₂₉, S₃₈₋₆₄, S₂₆₁₋₂₈₇, S₂₉₇₋₃₂₃, S₈₅₇₋₈₈₃, S₉₇₅₋₁₀₀₁, S₁₀₀₀₋₁₀₂₆, S₄₄₇₋₄₇₃, S₄₅₁₋₄₇₇, S₄₅₆₋₄₈₂, S₅₅₈₋₅₈₄, S₅₇₉₋₆₀₅, S₇₈₈₋₈₁₄, S₈₀₉₋₈₃₅, S₂₂₋₅₁, S₃₅₋₆₄, S₇₆₋₁₀₅, S₉₈₋₁₂₇, S₂₅₃₋₂₈₂, S₃₉₁₋₄₂₀, S₆₈₃₋₇₁₂, S₇₀₁₋₇₃₀, S₈₉₃₋₉₉₂, S₈₉₈₋₇₂₉, S₁₀₉₁₋₁₁₂₀, wherein a reference sequence is SEQ ID NO: 39 of S protein, and further wherein the antigen/epitope optionally comprises at least one amino acid mutation selected from the group consisting of L5F, S12F, S13I, L18F, T19R, T20N, P26S, Q52R, A67V, DEL69/70, V70F, G75V, T76I, D80G, D80A, T95I, D138Y, DEL141/143, G142D, DEL144/145, Y144S, Y145N, W152R, W152C, E154Q, E154K, DEL157/158, F157S, R190S, D215G, A222V, DEL241/243, DEL243/244, DEL247/253, D253G, W258L, Y265C, R346K, R346S, K417N, K417T, N439K, L452R, L452Q, S477N, T478K, E484K, E484Q, F490S, N501Y, K558N, A570D, D614G, H655Y, Q677H, P681R, P681H, A688V, A701V, T716L, D796H, S813N, T859N, F888L, A899S, D950N, D950H, Q957R, S982A, T1027I, Q1071H, E1072K, E1092K, H1101Y, H1101D, D1118H, I1130V, D1139H and V1176F;
(b) E₄₅₋₇₄, wherein a reference sequence is SEQ ID NO: 44 of E protein, and further wherein the antigen/epitope optionally comprises at least one amino acid mutation selected from the group consisting of L21F, S68F and P71L;
(c) M₃₄₋₄₈, M₃₉₋₅₃, M₄₀₋₅₄, M₉₅₋₁₀₉, M₉₉₋₁₁₃, M₃₄₋₅₄, M₆₅₋₈₅, M₉₃₋₁₁₃, M₅₉₋₈₅, M₉₅₋₁₂₁, M₉₃₋₁₂₂, wherein a reference sequence is SEQ ID NO: 45 of M protein, and further wherein the antigen/epitope optionally comprises at least one amino acid mutation selected from the group consisting of L29F, A63T, I82T, I82S and H125Y; and
(d) N₁₀₄₋₁₁₁₈, N₂₆₄₋₂₇₈, N₃₀₂₋₃₁₆, N₃₁₀₋₃₂₄, N₃₂₂₋₃₃₆, N₃₂₆₋₃₄₀, N₃₈₉₋₄₀₃, N₁₀₄₋₁₂₄, N₁₄₇₋₁₆₇, N₃₀₄₋₃₂₄, N₃₁₀₋₃₃₀, N₃₂₀₋₃₄₀, N₃₂₈₋₃₄₈, N₃₈₈₋₄₀₈, N₁₀₂₋₁₂₈, N₃₀₁₋₃₂₇, N₃₀₅₋₃₃₁, N₃₁₀₋₃₃₆, N₃₂₄₋₃₅₀, N₃₈₃₋₄₀₉, N₃₆₋₆₅, N₂₂₅₋₂₈₄, N₂₉₀₋₃₁₉, N₃₈₄₋₄₁₃, wherein a reference sequence is SEQ ID NO: 46 of N protein, and further wherein the antigen/epitope optionally comprises at least one amino acid mutation selected from the group consisting of D3L, DEL3, D3Y, A12G, P13L, D63G, P67S, P80R, A119S, I157V, P199L, S202R, R203M, R203K, G204R, G204P, T205I, DEL209, G212V, G214C, G215C, M234I, S235F, K256R, T366I and D377Y.

An antigen may be a single antigen or an antigenic fragment thereof, or a fusion antigen comprising at least two antigenic polypeptides fused together with or without a linker between the two antigenic polypeptides.

A fusion antigen may have a rigid linker, (EAAAAK)ₙ, connecting two different antigenic polypeptides, wherein n is an integer from 0-12, preferably from 2-6, more preferably from 3-4. In other words, the rigid linker comprises 0 to 12 repeats, 2 to 6 repeats or 3 to 4 repeats of the sequence EAAAAK (SEQ ID NO: 38).

The fusion protein of the invention may further comprise a rigid linker between the CD40-binding domain and the furin and/or cathepsin L cleavage site. The rigid linker comprises 0 to 12 repeats of the amino acid sequence EAAAAK (SEQ ID NO: 38). The rigid linker may be (EAAAAK)ₙ, or (SEQ ID NO: 38)ₙ, wherein n is an integer from 0-12, preferably from 2-6, more preferably from 3-4. In one embodiment, the rigid linker comprises 2 to 6 repeats or 3 to 4 repeats of SEQ ID NO: 38.

In another embodiment, the fusion protein of the invention comprises, or consists substantially of, an amino acid sequence that is at least 90%, 95% or 99% identical to SEQ ID NO: 47, 48, 49, 50, 51, 52, 53 or 54. In another embodiment, the fusion protein comprises, or consists substantially of, an amino acid sequence selected from the group consisting of SEQ ID NO: 47, 48, 49, 50, 51, 52, 53 and 54.

### Pharmaceutical Composition/Vaccine

The invention further relates to a pharmaceutical composition or a vaccine, which comprises:
(a) the fusion protein of the invention; and (b) a pharmaceutical acceptable carrier and/or an adjuvant.

A vaccine according to the invention is a prophylactic and/or therapeutic vaccine.

The term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329).

Suitable adjuvants include, but not limited to, a saponin-based adjuvant and a Toll-like receptor (TLR) agonist adjuvant. The saponin-based adjuvant may be GPI-0100, Quil A or QS-21. The TLR agonist adjuvant may be selected from a TLR3, TLR4 or TLR9 agonist, e.g., Poly I:C (TLR3 agonist), monophosphoryl lipid A (MPL; TLR4 agonist) or CpG oligonucleotide (TLR9 agonist). The CpG oligonucleotide adjuvant includes, but not limited to, class A CpG (i.e., CpG1585, CpG2216 or CpG2336), class B CpG (i.e., CpG1668, CpG1826, CpG2006, CpG2007, CpG BW006 or CpG D-SL01) and class C CpG (i.e., CpG2395, CpG M362 or CpG D-SL03). Another suitable CpG adjuvant is CpG1018 (Dynavax). In one embodiment, an adjuvant is a CpG oligonucleotide.

The pharmaceutical composition/vaccine may be an enteral or a parenteral dosage form, suitable for transdermal, transmucosal, nasopharyngeal, pulmonary or direct injection, or for systemic (e.g., parenteral) or local (e.g., intratumor or intralesional injection) administration. Parenteral injection may be via intravenous (i.v.), intraperitoneal (i.p.), intramuscular (i.m.), subcutaneous (s.c.) or intradermal (i.d.) routes. The pharmaceutical composition may also be administered orally, e.g., in the form of tablets, coated tablets, dragees, hard and soft gelatin capsules.

The dosage of the fusion protein may vary, depending on the disease to be controlled, the age and the individual condition of the patient and the mode of administration. The dosage may be fitted to individual requirements in each particular case so as to obtain a therapeutically effective amount of the fusion protein of the invention to achieve a desired therapeutic response.

For adult patients, a single dosage of about 0.1 to 50 mg, especially about 0.1 to 5 mg, comes into consideration. Depending on severity of the disease and the precise pharmacokinetic profile, the fusion protein may be administered with one dosage unit per week, bi-week or month, and totally give 1 to 6 dosage units per cycle to satisfy such treatment.

In one embodiment, the invention provides a kit or a packaged pharmaceutical composition comprising a fusion protein of the invention and instructions for using the fusion protein to treat one or more symptoms caused by SARS-CoV2 infection in a subject in need thereof.

### Combination Therapies

Further in another embodiment, the pharmaceutical composition of the invention further comprises an immune checkpoint antibody capable of activating a T cell.

In another aspect, the invention relates to a combination or a pharmaceutical composition comprising: (a) an immune checkpoint antibody capable of activating T cell; and (b) a fusion protein according to the invention. An immune checkpoint antibody includes, but not limited to, an anti-PD-1 antagonist antibody, or an anti-CD137 agonist antibody.

In another aspect, the invention relates to a combination or a pharmaceutical composition for use in eliciting an antigen-specific cell-mediated immune response, treating one or more symptoms of the disease caused by SARS-CoV2 in a subject in need thereof.

In one embodiment, the immune checkpoint antibody is an anti-PD-1 antibody, e.g., KEYTRUDA® (pembrolizumab), OPDIVO® (nivolumab), LIBTAYO® (cemiplimab), or JEMPERLI® (dostarlimab). In one embodiment, the immune checkpoint antibody is an anti-PD-L1 antibody, e.g., TECENTRIQ® (atezolizumab), IFINZI® (durvalumab), or BAVENCIO® (avelumab). In one embodiment, the immune checkpoint antibody is an anti-CTLA4 antibody, e.g., YERVOY® (ipilimumab). In one embodiment, the immune checkpoint antibody is an anti-LAG3 antibody, e.g., Relatlimab (BMS-986016). In one embodiment, the immune checkpoint antibody is an anti-TIGIT antibody, e.g., tiragolumab. In one embodiment, the immune checkpoint antibody is an anti-CD 137 antibody, e.g., LVGN6051 or Urelumab (BMS-663513). In one embodiment, the immune checkpoint antibody is an anti-OX40 antibody, e.g., PF-04518600, BMS-986178, or MEDI6469. In one embodiment, the immune checkpoint antibody is a CD137/PD-L1 bispecific antibody, e.g., FS222. In one embodiment, the immune checkpoint antibody is a CD137/OX40 bispecific antibody, e.g., FS120.

In another embodiment, the immune checkpoint antibody comprises a fully antibody, a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab₂, a minibody, or an antibody analogue comprising one or more CDRs.

### EXAMPLES

### Methods and Materials

Table 1 shows SEQ ID numbers of corresponding polypeptides and fusion proteins.

TABLE 1

| SEQ ID No. | Component name or sequence (N→C) | Length (aa) |
|---|---|---|
| 1 | Cleavable linker 1 | 4 |
| | RX¹X²R, wherein X¹ and X² are any amino acid residue. | |
| 2 | Cleavable linker 2 | 6 |
| | RX¹RX²X³R, wherein X¹ and X² are any amino acid | |
| | residue, and X³ is K, F or R. | |
| 3 | Rigid linker 1 (EAAAAK)₃ | 18 |
| 4 | Full length PE | 613 |
| 5 | PE translocation peptide (PE₂₈₀₋₃₀₅, minimal) | 26 |
| 6 | PE translocation peptide (PE₂₈₀₋₃₁₃) | 34 |
| 7 | PE translocation peptide (PE₂₆₈₋₃₁₃) | 46 |
| 8 | PE translocation peptide (PE₂₅₃₋₃₁₃) | 61 |
| 9 | PE translocation peptide (PE₂₅₃₋₃₆₄) | 112 |
| 10 | Full length Shiga toxin (Stx) subunit A | 293 |
| 11 | Full length Shiga-like toxin I (Slt-I) subunit A | 293 |
| 12 | Stx translocation peptide (Stx₂₄₀₋₂₄₇, minimal) | 8 |
| 13 | Stx translocation peptide (Stx₂₄₀₋₂₅₁) | 12 |
| 14 | Stx translocation peptide (Stx₂₁₁₋₂₄₇) | 37 |
| 15 | Stx translocation peptide (Stx₂₁₁₋₂₅₁) | 41 |
| 16 | Stx translocation peptide (Stx₁₆₈₋₂₅₁) | 84 |
| 17 | Full length CD40 ligand (CD40L₁₋₂₆₁) | 261 |
| 18 | Truncated CD40 ligand (CD40L₄₇₋₂₆₁) | 215 |
| 19 | Truncated CD40 ligand (CD40L₁₀₈₋₂₆₁; i.e., 18sCD40L) | 154 |
| 20 | Anti-CD40 scFv (V_{H}-L-V_{L}) | 246 |
| 21 | Anti-CD40 scFv (V_{L}-L-V_{H}) | 246 |
| 22 | V_{H} of the anti-CD40 scFv | 119 |
| 23 | V_{L} of the anti-CD40 scFv | 112 |
| 24 | V_{H} CDR1 GFTFSTYGMH | 10 |
| 25 | V_{H} CDR2 GKGLEWLSYISGGSSYIFYADSVRGR | 26 |
| 26 | V_{H} CDR3 CARILRGGSGMDL | 13 |
| 27 | V_{L} CDR1 CTGSSSNIGAGYNVY | 15 |
| 28 | V_{L} CDR2 GNINRPS | 7 |
| 29 | V_{L} CDR3 CAAWDKSISGLV | 12 |
| 30 | ER retention sequence KDEL | 4 |
| 31 | ER retention sequence KKDLRDELKDEL | 12 |
| 32 | ER retention sequence KKDELRDELKDEL | 13 |
| 33 | ER retention sequence KKDELRVELKDEL | 13 |
| 34 | ER retention sequence KDELKDELKDEL | 12 |
| 35 | CD28 consensus sequence | 28 |
| | T¹D²I³Y⁴F⁵C⁶K⁷X⁸E⁹X¹⁰X¹¹Y¹²P¹³P¹⁴P¹⁵Y¹⁶X¹⁷D¹⁸N¹⁹ | |
| | E²⁰K²¹S²²N²³G²⁴T²⁵I²⁶I²⁷H²⁸, wherein X⁸ is | |
| | I or L, X¹⁰ is V, F or A, X¹¹ is M or L, X¹⁷ is L or I. | |
| 36 | CD28-activating peptide (minimal) | 28 |
| 37 | CD28-activating peptide | 53 |
| 38 | Rigid linker EAAAAK | 6 |
| 39 | SARS-CoV2 spike (S) protein | 1273 |
| | (NCBI accession number: YP_009724390.1) | |
| 40 | S1 subunit of S protein | 672 |
| | (Corresponding to 14-685 residues of SEQ ID NO: 39) | |
| 41 | Receptor-binding domain of S protein (S_{RBD}) | 223 |
| | (Corresponding to 319-541 residues of SEQ ID NO: 39) | |
| 42 | Truncated S protein (S₃₁₉₋₆₈₅) | 367 |
| | (Corresponding to 319-685 residues of SEQ ID No. 39) | |
| 43 | S2 subunit of S protein | 588 |
| | (Corresponding to 686-1273 residues of SEQ ID NO: 39) | |
| 44 | SARS-CoV2 envelope (E) protein | 75 |
| | (NCBI accession number: YP_009724392.1) | |
| 45 | SARS-CoV2 membrane (M) protein | 222 |
| | (NCBI accession number: YP_009724393.1) | |
| 46 | SARS-CoV2 nucleocapsid (N) protein | 419 |
| | (NCBI accession number: YP_009724397.2) | |
| 47 | Fusion protein CD40L-T^{PE}-S_{RBD} | 462 |
| 48 | Fusion protein CD40L-T^{PE}-S₃₁₉₋₆₈₅ | 606 |
| 49 | Fusion protein CD40L-T^{PE}-E/M | 536 |
| 50 | Fusion protein CD40L-T^{PE}-N | 658 |
| 51 | Fusion protein S_{RBD}-T^{Stx}-CD40L | 469 |
| 52 | Fusion protein S₃₁₉₋₆₈₅-T^{Stx}-CD40L | 613 |
| 53 | Fusion protein E/M-T^{Stx}-CD40L | 543 |
| 54 | Fusion protein N-T^{Stx}-CD40L | 665 |

Flow cytometry. Splenocytes were stimulated with an antigenic stimulator (for boosting an immune response against the specific antigen used in the fusion protein of the invention) for 2 hours at 37° C., followed by treating with 50 μg/mL of Brefeldin A and Monensin at 37° C. for 2 hours. The cells were harvested, washed with PBS containing 0.5% BSA, and stained with APC/Cy7-conjugated anti-CD3 antibody, PerCP/Cy5.5-conjugated anti-CD4 antibody, FITC-conjugated anti-CD8 antibody, PE-conjugated anti-CD44 antibody and APC-conjugated anti-CD62L antibody simultaneously. After wash, the cells were permeabilized, fixed and intracellularly stained with PE-conjugated anti-IFN-7 antibody and PE/Cy7-conjugated anti-IL-2 antibody and eFluor450-conjugated anti-TNF-α antibody simultaneously. The intracellular cytokine induction (IFN-γ, IL-2 or TNF-α) of splenocytes with CD8+ or CD4+ memory T cell phenotypes (CD3⁺CD44^{hi}CD62L^{lo}) were further analyzed by Gallios flow cytometer and Kaluza software.

Enzyme-linked immunospot (ELISpot) assay. Splenocytes were seeded in triplicate in a pretreated murine IFN-γ capturing 96-well plate (CTL IMMUNOSPOT®) at a cell density of 2×10⁵ cells/well in the presence or absence of an antigenic stimulator (for boosting an immune response). The cells were discarded after 24 hours of incubation at 37° C. After wash, the captured IFN-γ was detected by biotin-conjugated anti-murine IFN-γ antibody at room temperature for 2 hours and the IFN-γ-immunospots were developed according to the manufacturer's instructions. The scanning and counting of IFN-γ-immunospots was performed by IMMUNOSPOT® S5 Micro analyzer (CTL). The results were presented as IFN-γ immunospots per million splenocytes.

Indirect enzyme-linked immunosorbent assay (ELISA). Collected whole blood samples were left undisturbed at 4° C. for 30-60 minutes followed by centrifugation at 5,000 g for 10 minutes to pellet the clot. The serum samples were stored at −20° C. To capture desired antigen-specific antibody, the recombinant antigen corresponding to the antigen in said fusion protein was synthesized and used as a coating protein. The coating protein was diluted in guanidine coating buffer (2 M guanidine hydrochloride, 500 mM Na₂HPO₄, 25 mM citrate, pH 4.0-4.4) and distributed into 96-well plate at 1 μg/well. After overnight incubation at 4° C., the 96-well plate was blocked with 1% BSA in PBS at 37° C. for 1 hour. The serum samples were thawed, and subsequently 10-fold serial diluted in PBS with 1% BSA. The coated protein was incubated with 100 μl of 1000-fold diluted serum sample at 37° C. for 2 hours. After 4 times washing with phosphate buffered saline TWEEN®-20 (PBST), the antigen-specific antibodies which bound to coating proteins were detected by horseradish peroxidase (HRP)-conjugated goat anti-mouse IgG (at a dilution of 1:10,000, Cat #31430, Thermo Fisher Science) at 37° C. for 30 minutes. Following 4 times of washing with PBST, the HRP-mediated color development was catalyzed in the presence of 100 μL of TMB substrate and quenched by 100 μL of 1 N HCl. The relative titers of antigen-specific antibody in the serum samples were determined by the absorbance at 450 nm.

Statistical analysis. Using 1-test, results considered significant when p<0.05.

### Example 1

### Construction of Expression Vectors

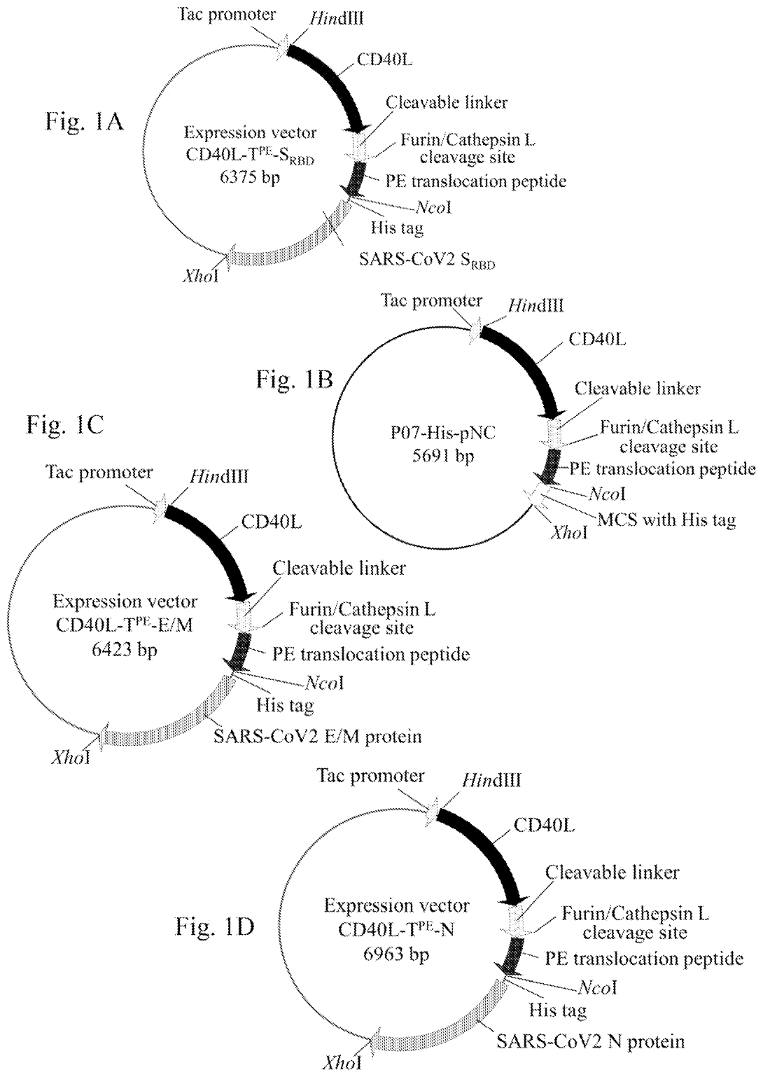

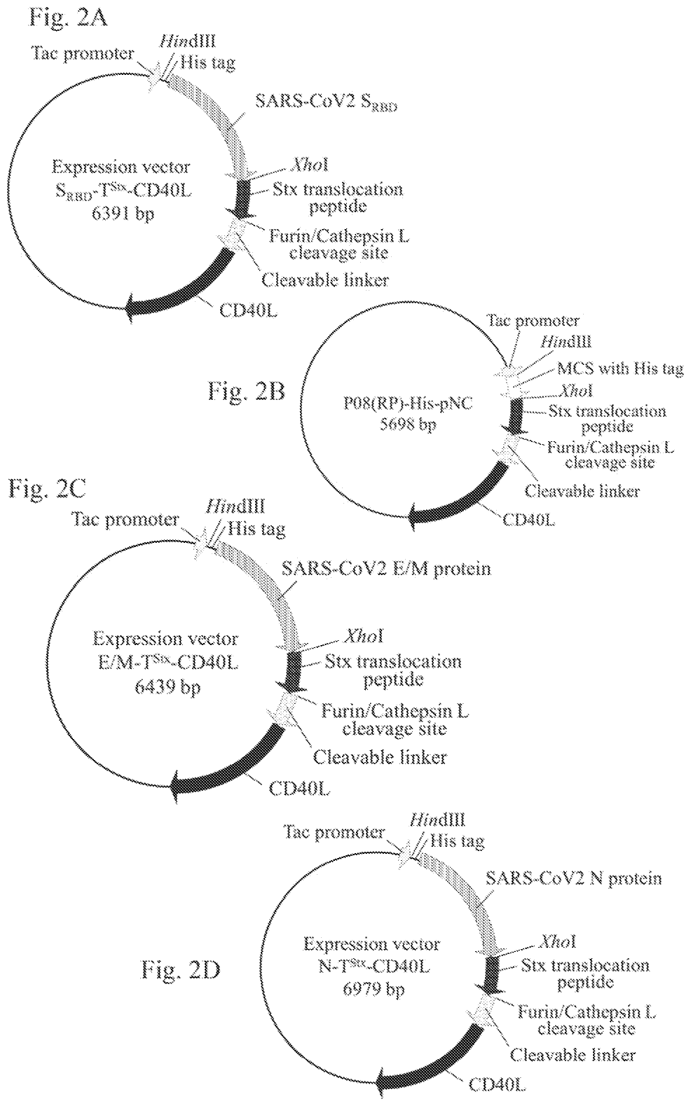

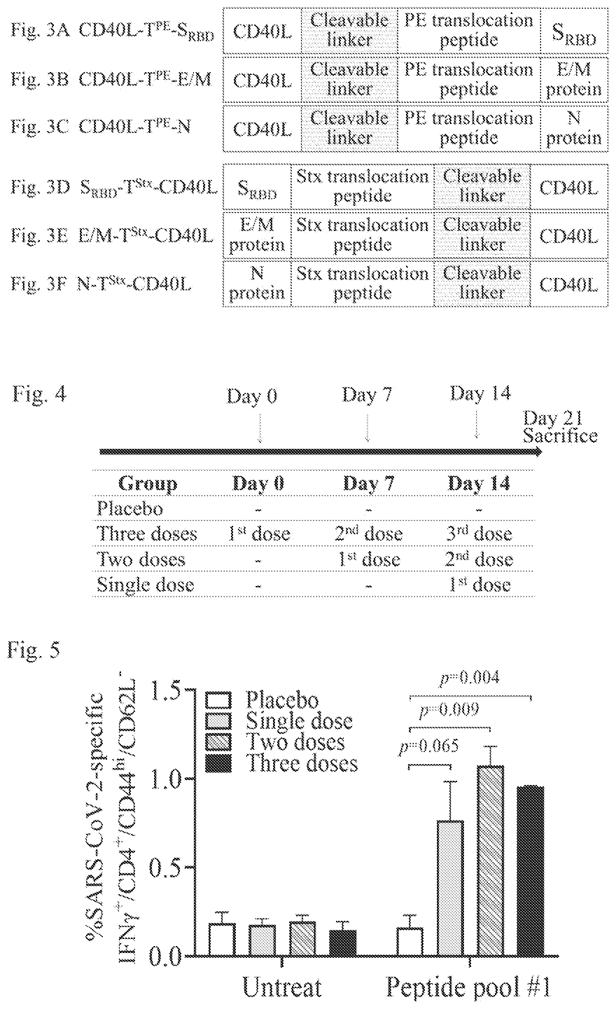

CD40L-T^{PE}-S_{RBD}, CD40L-T^{PE}-E/M and CD40L-T^{PE}-N. The vector CD40L-T^{PE}-S_{RBD} (FIG. 1A) was constructed to generate CD40L-T^{PE}-S_{RBD} (SEQ ID NO: 47; FIG. 3A) fusion protein, which comprises: (a) a truncated CD40 ligand CD40L₁₀₈₋₂₆₁ (SEQ ID NO: 19); (b) a cleavable peptide linker, comprising (EAAAAK)₃ (SEQ ID NO: 3) and RX¹RX²X³R (SEQ ID NO: 2; wherein X¹ is A, X² is Y, X³ is K); (c) a PE translocation peptide PE₂₈₀₋₃₀₅ (SEQ ID NO: 5); and (d) a receptor-binding domain of SARS-CoV2 spike protein (S_{RBD}; SEQ ID NO: 41) used as an antigen. FIGS. 3A-F illustrates various embodiments of the fusion protein according to the invention.

Briefly, a DNA fragment encoding ^{HindIII}CD40L-Linker-PE^{NcoI,XhoI,SalI}, which comprises the CD40L₁₀₈₋₂₆₁, the cleavable linker and the PE translocation peptide (PE₂₈₀₋₃₀₅), was PCR synthesized, digested by HindIII/SalI and ligated into the plasmid pTAC-MAT-Tag-2 (Catalog No. E5405, Sigma-Aldrich) having HindIII/XhoI cutting sites to obtain the plasmid P07-His-pNC (FIG. 1B). Another DNA fragment encoding said antigen SARS-CoV2 S_{RBD} carrying a His tag was inserted into the plasmid P07-His-pNC (FIG. 1B) via restriction enzymes NcoI/XhoI to generate the expression vector CD40L-T^{PE}-S_{RBD} (FIG. 1A).

The cleavable linker allows furin and/or cathepsin L protease to cut the fusion protein of the invention for releasing the T^{PE}-S_{RBD} fragment from the fusion protein.

Using a similar method described above, any other antigen(s) of interest from SARS-CoV2 may replace the antigen S_{RBD} and be inserted into the plasmid of FIG. 1B to generate an expression vector like FIG. 1A for expressing a fusion protein comprising the antigen(s) of interest.

Similarly, an expression vector (FIG. 1C) for generating CD40L-T^{PE}-E/M (SEQ ID NO: 49; FIG. 3B) fusion protein was constructed by replacing the antigen S_{RBD} with a fusion antigen E/M consisting of a SARS-CoV2 envelope (E) protein (SEQ ID NO: 44) and a SARS-CoV2 membrane (M) protein (SEQ ID NO: 45). Another expression vector (FIG. 1D) for generating CD40L-T^{PE}-N (SEQ ID NO: 50; FIG. 3C) fusion protein was constructed similarly by replacing the antigen S_{RBD} with a SARS-CoV2 nucleocapsid (N) protein (SEQ ID NO: 46).

S_{RBD}-T^{Stx}-CD40L, E/M-T^{Stx}-CD40L, and N-T^{Stx}-CD40L. The vector S_{RBD}-T^{Stx}-CD40L (FIG. 2A) was constructed to generate S_{RBD}-T^{Stx}-CD40L (SEQ ID NO: 51; FIG. 3D) fusion protein, which comprise: (a) a receptor-binding domain of SARS-CoV2 spike protein (S_{RBD}; SEQ ID NO: 41) used as an antigen, (b) a Stx translocation peptide Stx₂₁₁₋₂₄₇ (SEQ ID NO: 14), (c) a cleavable peptide linker, comprising RX¹X²R (SEQ ID NO: 1; wherein X¹ is V, X² is A) and (EAAAAK)₃ (SEQ ID NO: 3), and (d) a truncated CD40 ligand CD40L₁₀₈₋₂₆₁ (SEQ ID NO: 19).

Briefly, a DNA fragment encoding ^{HindIII,XhoI}Stx-Linker-CD40L^{SalI}, which comprises the Stx translocation peptide (Stx₂₁₁₋₂₄₇), the cleavable linker and the CD40L₁₀₈₋₂₆₁, was PCR synthesized, digested by HindIII/SalI, and ligated into plasmid pTAC-MAT-Tag-2 backbone having HindIII/XhoI cutting sites to obtain the plasmid P08(RP)-His-pNC (FIG. 2B). Another DNA fragment encoding said antigen SARS-CoV2 S_{RBD} carrying a His tag was inserted into the plasmid P08(RP)-His-pNC (FIG. 2B) via restriction enzymes HindIII/XhoI to generate the expression vector S_{RBD}-T^{Stx}-CD40L (FIG. 2A).

The cleavable linker is vital for the fusion protein of the invention because it allows the fusion protein to be cut by furin and/or cathepsin L protease to release the S_{RBD}-T^{Stx} fragment from the fusion protein.

Using a similar method described above, any other antigen(s) of interest from SARS-CoV2 may replace the antigen S_{RBD} and be inserted into the plasmid of FIG. 2B to generate an expression vector like FIG. 2A for expressing a fusion protein comprising the antigen(s) of interest.

Similarly, an expression vector (FIG. 2C) for generating E/M-T^{Stx}-CD40L (SEQ ID NO: 53; FIG. 3E) fusion protein was constructed similarly by replacing the antigen S_{RBD} with a fusion antigen E/M consisting of a SARS-CoV2 envelope (E) protein (SEQ ID NO: 44) and a SARS-CoV2 membrane (M) protein (SEQ ID NO: 45). Another expression vector (FIG. 2D) for generating N-T^{Stx}-CD40L (SEQ ID NO: 54; FIG. 3F) fusion protein was constructed similarly by replacing the antigen S_{RBD} with a SARS-CoV2 nucleocapsid (N) protein (SEQ ID NO: 46).

### Example 2

### Protein Expression

E. coli BL21 cells harboring the protein expression vector N-T^{Stx}-CD40L were grown in ZY media (10 g/L tryptone and 5 g/L yeast extract) containing a selection antibiotic at 37° C. When the culture reached an early log phase, (OD₆₀₀=2 to 5), the expression of fusion protein was induced by isopropyl-1-thio-β-D-galactopyranoside (IPTG) (0.5 to 2 mM). Cells were harvested after 4 hours of IPTG induction and disrupted by sonication. The inclusion bodies were isolated and solubilized in solubilization buffer (6 M guanidine hydrochloride, 20 mM potassium phosphate, 500 mM NaCl, 20 mM imidazole, 1 mM DTT, pH 7.4) to recover overexpressed fusion proteins. After purification, the refolding of the fusion protein was performed by dialysis against 20- to 50-fold volume of dialysis buffer (10 mM PBS) at 4° C. overnight. The refolded fusion proteins were subject to SDS-PAGE analyses under reduced (with dithiothreitol; +DTT) and non-reduced (without dithiothreitol; −DTT) conditions to evaluate whether they were properly refolded.

The fusion proteins CD40L-T^{PE}-S_{RBD}, CD40L-T^{PE}-E/M, CD40L-T^{PE}-N, S_{RBD}-T^{Stx}-CD40L and E/M-T^{Stx}-CD40L are expressed, purified and refolded by using the same method described above. The N-T^{Stx}-CD40L was chosen as a representative of the fusion proteins according to the invention and further subjected to various tests to evaluate immunogenicity and therapeutic effects.

### Example 3

Immunogenicity Analyses of the Fusion Proteins. C57BL/6JNarl female mice at 5-weeks-old age were randomly divided into four groups (n=5 per group): (1) placebo group; (2) three doses-group; (3) two doses-group; and (4) one single dose group. Mice in the placebo group were injected s.c. with PBS on Days 0, 7 and 14. Mice in three doses-group were vaccinated on Days 0, 7 and 14, mice in the two doses-group vaccinated on Days 7 and 14, and mice in the one single dose group vaccinated with the fusion protein on Day 14. To vaccinate mice, N-T^{Stx}-CD40L fusion protein (100 μg) was adjuvanted with CpG1826 ODN (50 μg), and injected s.c. according to the dosing schedule shown in FIG. 4. Blood samples were collected on Days 0, 7, 14 and 21. On Day 21, the animals were sacrificed and splenocytes were harvested and cultured.

The splenocytes were used to analyze intracellular cytokine (IFN-γ and IL-2) induction in the CD4⁺ memory T cells by using flow cytometry. Additionally, the frequency of IFN-γ-secreting splenocytes was analyzed using Enzyme-linked immunospot (ELISpot) assay. The levels of serum antigen-specific antibody in the blood samples were analyzed by using ELISA.

FIG. 5 shows the result of IFN-γ induction after the splenocytes being stimulated with or without an antigenic stimulator (a 32 a.a. peptide pool covering the entire SARS-CoV2 N protein). The cytokine induction of IFN-γ in CD4⁺ memory T cells from the animals receiving three doses or two doses of N-T^{Stx}-CD40L showed a significant increase as compared to the placebo group. Noticeably, one dose of N-T^{Stx}-CD40L fusion protein was sufficient to induce potent IFN-γ positive CD4⁺ memory T cells.

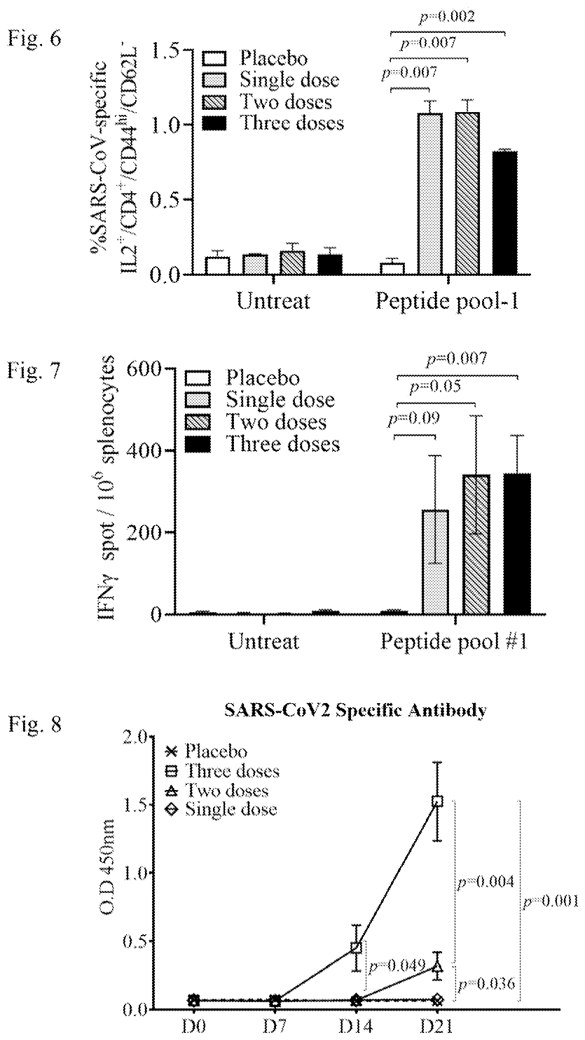

FIG. 6 shows the results of IL-2 induction after the splenocytes being stimulated with or without an antigenic stimulator (a 32 a.a. peptide pool as aforementioned). The profile of IL-2 induction was like that of IFN-γ induction. The IL-2 cytokine inductions in CD4⁺ memory T cells from the N-T^{Stx}-CD40L fusion protein-vaccinated animal groups (three doses, two doses or one dose) significantly increased as compared to the placebo group. Noticeably, one single dose of the fusion protein was sufficient in inducing potent IL-2 positive CD4⁺ memory T cells.

FIG. 7 shows the results of IFN-γ immunospots after the splenocytes being stimulated with or without an antigenic stimulator as aforementioned in vitro. The IFN-γ-secreting splenocytes from the N-T^{Stx}-CD40L fusion protein-vaccinated animals (three doses, two doses and one dose) significantly increased as compared to the placebo group. One single dose of the fusion protein was sufficient in inducing a significant increase in IFN-γ-secreting splenocytes. The result was consistent with the finding above in IFN-γ positive CD4⁺ memory T cells.

In the animals vaccinated with three doses of N-T^{Stx}-CD40L fusion protein, the N protein-specific antibody levels started to increase significantly after the second shot (Day 7) and further increased after the third shot (Day 14) of the fusion protein. On Day 21, the serum SARS-CoV2 specific antibody levels in the group vaccinated with three doses were the highest among the animal groups (FIG. 8).

Similarly, the fusion proteins CD40L-T^{PE}-S_{RBD}, CD40L-T^{PE}-F/M, CD40L-T^{PE}-N, S_{RBD}-T^{Stx}-CD40L and E/M-T^{Stx}-CD40L are to be subjected to immunogenicity analyses. Based on the same mechanism of action, the antigen-carrying fusion proteins of the invention are expected to induce potent immune responses, such as inducing antigen-specific antibody, T cell activation, and inducing IFN-γ, IL-2 and TNF-α.

One can reasonably conclude that a fusion protein of the invention is effective in eliciting a potent T cell immune response, increasing IFN-γ and IL-2 expressions, and generating a SARS-CoV2 antigen-specific antibody response.

### Example 4

Preventive Effect of the Fusion Proteins in Hamster Model. N-T^{Stx}-CD40L fusion protein was tested in hamster model to evaluate the preventive effect on viral infection. Female Golden Syrian Hamsters (n=5) of 6- to 7-weeks-old were injected s.c. with PBS (placebo), a high dose (200 μg) or a low dose (100 μg) of the fusion protein adjuvanted with CpG1826 ODN (50 μg) on day 0 and day 7, then challenged with 1×10⁴ TCID50 of SARS-CoV2 variant B.1.617.2 (Delta) (100 μl, intranasally) on day 14. Body weights of hamsters were recorded daily after the infection. On the 3^{rd} day after the viral challenge, hamsters were sacrificed. The superior lobe of left lung was fixed in 10% paraformaldehyde for histopathological examinations and the rest of lung was collected for viral load determination (TCID50 assay).

To perform TCID50 assay, the middle, inferior, and post-caval lung lobes from the animals were homogenized in 4 ml of DMEM containing 2% FBS and 1% penicillin/streptomycin. The tissue homogenate was centrifugated and the supernatant collected for live virus titration. Ten-fold serial dilutions of each sample were added onto Vero E6 cell monolayer in duplicate and incubated for 4 days, and cells were observed by microscope daily. The plates were washed with tap water and scored for infection. The fifty-percent tissue culture infectious dose (TCID50)/ml was calculated by the Reed and Muench method.

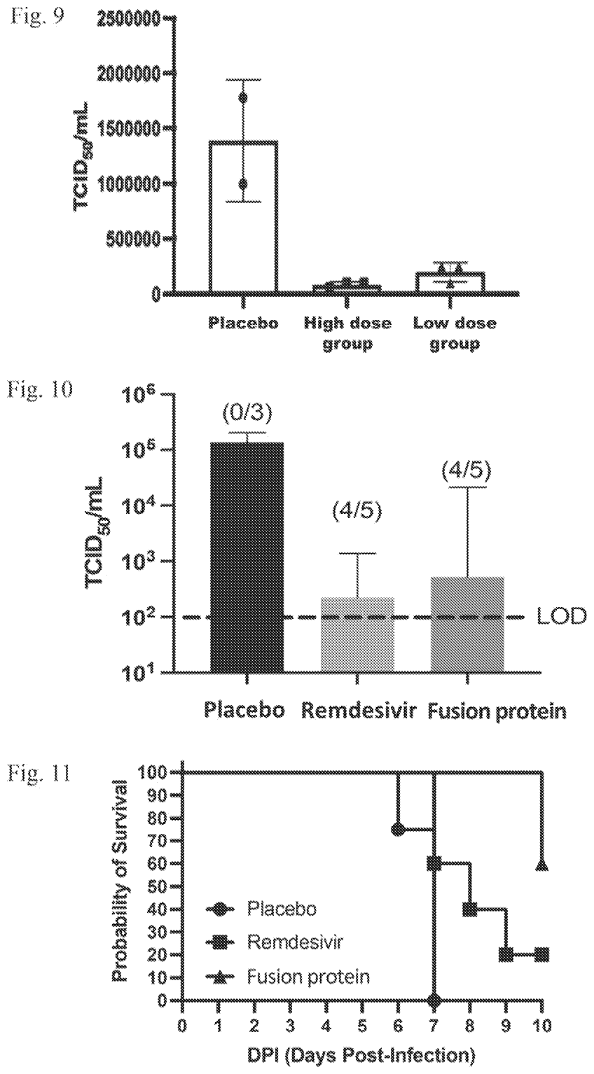

FIG. 9 shows the level of lung viral titer from animals receiving different doses of N-T^{Stx}-CD40L fusion protein. The viral titers in the high dose and low dose groups were significantly reduced as compared to the placebo. The results indicated that the fusion protein of the invention could potently reduce and inhibit SARS-CoV2 viral infection.

The fusion proteins CD40L-T^{PE}-S_{RBD}, CD40L-T^{PE}-E/M, CD40L-T^{PE}-N, S_{RBD}-T^{Stx}-CD40L and E/M-T^{Stx}-CD40L are to be tested in the aforementioned hamster model. On the basis of the mechanism of actions of the fusion proteins of the invention, these antigen-carrying fusion proteins are expected to effectively reduce and inhibit SARS-CoV2 viral infection in the hamster model.

### Example 5

Therapeutic Effect of the Fusion Proteins in hACE2 transgenic moue model. N-T^{Stx}-CD40L fusion protein was tested in hACE2 transgenic mice to evaluate the therapeutic effect of the fusion protein of the invention. Briefly, male K18-hACE2 transgenic C57BL/6J mice at 8-weeks-old age from Jackson Laboratory were challenged with 1×10³ PFU of SARS-CoV2 variant B.1.617.2 (Delta) two hours before treatment on day 0. After the viral challenge, mice were grouped and treated with (A) placebo (n=3); (B) remdesivir (1 mg/dose; n=5); and (C) the fusion protein (200 μg/dose; n=5), respectively. Placebo (PBS injected s.c.) and Remdesivir (injected i.v.) were each given one dose daily from Day 0 to Day 6. The fusion protein N-T^{Stx}-CD40L was adjuvanted with CpG1826 ODN (50 μg) and injected s.c. on Day 0 and Day 7. Body weights of mice were recorded daily after the viral challenge. On the 12^{th} day after the viral infection, all the survived mice were sacrificed. The superior lobe of the left lung was fixed in 10% paraformaldehyde for histopathological examinations and the rest of the lung was collected for viral load determination using TCID50 assay.

FIG. 10 shows lung viral titers in SARS-CoV2-infected animal groups treated with placebo, remdesivir and N-T^{Stx}-CD40L fusion protein, respectively. The results indicated that both N-T^{Stx}-CD40L and remdesivir treatment significantly reduced lung viral loads in the treated mice as compared to the placebo group. Remdesivir is a first-line drug for treating SARS-CoV2 infection. In both fusion protein-treated and remdesivir-treated groups, each group had four mice out of five mice showing a low viral load, that is, below the level of detection (LOD). The results indicated that the fusion protein of the invention could effectively clean the viruses and exhibit a comparable or similar therapeutic effects as compared to remdesivir.

FIG. 11 shows the probability of survival in animal groups. The mice in the placebo group all died 7 days post-infection. The survival rate of the mice in the remdesivir-treated group dropped to 20% on Day 9 post-infection. Notably, mice in the fusion protein-treated group maintained 100% of the survival rate until Day 9 and the survival rate declined to 60% on Day 10 post-infection. The results indicated that the fusion protein of the invention performed better than remdesivir in increasing the survival rate of SARS-CoV2 infected animals.

The fusion proteins CD40L-T^{PE}-S_{RBD}, CD40L-T^{PE}-E/M, CD40L-T^{PE}-N, S_{RBD}-T^{Stx}-CD40L and E/M-T^{Stx}-CD40L are to be tested in the transgenic mouse model aforementioned. On the basis of the mechanism of actions of the fusion proteins of the invention, these antigen-carrying fusion proteins are expected to effectively reduce lung viral loads, increase survival rate and treat SARS-CoV2 infections.

All references cited and discussed in this specification are incorporated herein by reference in their entireties and to the same extent as if each reference was individually incorporated by reference.

What is claimed is:

1. A fusion protein comprising:
(a) a CD40-binding domain, which is a CD40 ligand (CD40L) or a functional fragment thereof comprising an amino acid sequence that is at least 95% identical to SEQ ID NO: 19, said CD40L or functional fragment thereof consisting of 154-261 amino acid residues in length;
(b) an antigen of severe acute respiratory syndrome coronavirus 2 (SARS-CoV2);
(c) a translocation domain, located between the CD40-binding domain and the antigen, said translocation domain being selected from the group consisting of:
(c1) a Shiga toxin (Stx) translocation peptide; and
(c2) a Pseudomonas Exotoxin A (PE) translocation peptide; and
(d) a furin and/or cathepsin L cleavage site, located between the CD40-binding domain and the translocation domain,
wherein when the translocation domain is the Stx translocation peptide, the antigen is located at the N-terminal of the fusion protein; and when the translocation domain is the PE translocation peptide, the CD40-binding domain is a CD40L monomer and located at the N-terminal of the fusion protein.

2. The fusion protein of claim 1, wherein the translocation domain is the Stx translocation peptide, the Stx translocation peptide consisting of 8-84 amino acid residues in length and comprising an amino acid sequence selected from the group consisting of SEQ ID NOs:12, 13, 14, 15 and 16.

3. The fusion protein of claim 1, wherein the translocation domain is the Stx translocation peptide consisting of 8-84 amino acid residues in length and comprises an amino acid sequence that is at least 95% identical to SEQ ID NOs: 12, 13, 14, 15 or 16.

4. The fusion protein of claim 1, wherein the translocation domain is the PE translocation peptide, the PE translocation peptide consisting of 26-112 amino acid residues in length and comprising an amino acid sequence of SEQ ID NO: 5.

5. The fusion protein of claim 1, wherein the translocation domain is the PE translocation peptide, the PE translocation peptide consisting of 26-112 amino acid residues in length and comprising an amino acid sequence that is at least 95% identical to SEQ ID NO: 5, 6, 7, 8 or 9.

6. The fusion protein of claim 1, wherein the furin and/or cathepsin L cleavage site comprises an amino acid sequence of SEQ ID NO: 1 or 2.

7. The fusion protein of claim 1, wherein the CD40 ligand (CD40L) or the functional fragment thereof comprises the amino acid sequence of SEQ ID NO: 19 with 154-261 amino acid residues in length.

8. The fusion protein of claim 1, wherein the CD40L consists of 154-261 amino acid residues in length and comprises an amino acid sequence that is at least 97% identical to SEQ ID NO: 17, 18 or 19.

9. The fusion protein of claim 1, wherein the SARS-CoV2 is a Wuhan-Hu-1 strain with a NCBI reference number NC_045512.2, or a viral variant thereof.

10. The fusion protein of claim 1, wherein the antigen comprises at least one polypeptide selected from spike (S) protein, envelope (E) protein, membrane (M) protein or nucleocapsid (N) protein of SARS-CoV2.

11. The fusion protein of claim 10, wherein the antigen comprises at least one polypeptide selected from the groups consisting of:
(a) a polypeptide of 8-1273 amino acid residues selected from a region of SEQ ID NO: 39 of S protein, wherein the polypeptide optionally comprises at least one amino acid mutation selected from the group consisting of L5F, S12F, S13I, L18F, T19R, T20N, P26S, Q52R, A67V, DEL69/70, V70F, G75V, T76I, D80G, D80A, T95I, D138Y, DEL141/143, G142D, DEL144/145, Y144S, Y145N, W152R, W152C, E154Q, E154K, DEL157/158, F157S, R190S, D215G, A222V, DEL241/243, DEL243/244, DEL247/253, D253G, W258L, Y265C, R346K, R346S, K417N, K417T, N439K, L452R, L452Q, S477N, T478K, E484K, E484Q, F490S, N501Y, K558N, A570D, D614G, H655Y, Q677H, P681R, P681H, A688V, A701V, T716L, D796H, S813N, T859N, F888L, A899S, D950N, D950H, Q957R, S982A, T1027I, Q1071H, E1072K, E1092K, H1101Y, H1101D, D1118H, I1130V, D1139H and V1176F;
(b) a polypeptide of 8-75 amino acid residues selected from a region of SEQ ID NO: 44 of E protein, wherein the polypeptide optionally comprises at least one amino acid mutation selected from the group consisting of L21F, S68F and P71L;
(c) a polypeptide of 8-222 amino acid residues selected from a region of SEQ ID NO: 45 of M protein, wherein the polypeptide optionally comprises at least one amino acid mutation selected from the group consisting of L29F, A63T, I82T, I82S and H125Y; and
(d) a polypeptide of 8-491 amino acid residues selected from a region of SEQ ID NO: 46 of N protein, wherein the polypeptide optionally comprises at least one amino acid mutation selected from the group consisting of D3L, DEL3, D3Y, A12G, P13L, D63G, P67S, P80R, A119S, I157V, P199L, S202R, R203M, R203K, G204R, G204P, T205I, DEL209, G212V, G214C, G215C, M234I, S235F, K256R, T366I and D377Y.

12. The fusion protein of claim 1, wherein the antigen is a fusion antigen comprising at least two polypeptides independently selected from S protein, E protein, M protein or N protein of the SARS-CoV2.

13. The fusion protein of claim 1, wherein the SARS-CoV2 is a viral variant comprising at least one amino acid mutation as follows:
(a) the at least one amino acid mutation in S protein selected from the group consisting of L5F, S12F, S13I, L18F, T19R, T20N, P26S, Q52R, A67V, DEL69/70, V70F, G75V, T76I, D80G, D80A, T95I, D138Y, DEL141/143, G142D, DEL144/145, Y144S, Y145N, W152R, W152C, E154Q, E154K, DEL157/158, F157S, R190S, D215G, A222V, DEL241/243, DEL243/244, DEL247/253, D253G, W258L, Y265C, R346K, R346S, K417N, K417T, N439K, L452R, L452Q, S477N, T478K, E484K, E484Q, F490S, N501Y, K558N, A570D, D614G, H655Y, Q677H, P681R, P681H, A688V, A701V, T716L, D796H, S813N, T859N, F888L, A899S, D950N, D950H, Q957R, S982A, T1027I, Q1071H, E1072K, E1092K, H1101Y, H1101D, D1118H, I1130V, D1139H and V1176F, wherein a reference sequence for the mutation is SEQ ID NO: 39 of the S protein;
(b) the at least one amino acid mutation in E protein, selected from the group consisting of L21F, S68F and P71L, wherein a reference sequence for the mutation is SEQ ID NO: 44 of the E protein;
(c) the at least one amino acid mutation in the M protein selected from the group consisting of L29F, A63T, I82T, I82S and H125Y, wherein a reference sequence for the mutation is SEQ ID NO: 45 of the M protein; and
(d) the at least one amino acid mutation in N protein, selected from the group consisting of D3L, DEL3, D3Y, A12G, P13L, D63G, P67S, P80R, A119S, I157V, P199L, S202R, R203M, R203K, G204R, G204P, T205I, DEL209, G212V, G214C, G215C, M234I, S235F, K256R, T366I and D377Y, wherein a reference sequence for the mutation is SEQ ID NO: 46 of the N protein.

14. A pharmaceutical composition comprising:
(a) the fusion protein of claim 1; and
(b) a pharmaceutical acceptable carrier and/or an adjuvant.

15. A method for eliciting an antigen-specific, cell-mediated immune response against SARS-CoV2 infection, and/or for reducing, inhibiting, treating, and ameliorating symptoms caused by SARS-CoV2 infection in a subject in need thereof, comprising:
administering a therapeutically effective amount of the fusion protein of claim 1 to the subject in need thereof, and thereby eliciting the antigen-specific, cell-mediated immune response against the SARS-CoV2 infection, and/or reducing, inhibiting, treating and ameliorating the symptoms caused by the SARS-CoV2 infection in the subject in need thereof.

16. The pharmaceutical composition of claim 14, further comprising an immune checkpoint antibody that can activate a T cell.

17. The pharmaceutical composition of claim 16, wherein the immune checkpoint antibody is selected from the group consisting of an anti-PD-L1 antibody, an anti-CTLA-4 antibody, an anti-LAG3 antibody, an anti-TIGIT antibody, anti-CD137 antibody, an anti-OX40 antibody, a CD137/PD-L1 bispecific antibody, and a CD137/OX40 bispecific antibody.

18. The fusion protein of claim 2, wherein the antigen is selected from the group consisting of a SARS-CoV2 nucleocapsid (N) protein, a fusion antigen E/M a SARS-CoV2 envelope (E) protein and a SARS-CoV2 membrane (M) protein, and a receptor-binding domain of SARS-CoV2 spike protein (S_{RBD}).

19. The fusion protein of claim 4, wherein the antigen is selected from the group consisting of a SARS-CoV2 nucleocapsid (N) protein, a fusion antigen E/M consisting of a SARS-CoV2 envelope (E) protein and a SARS-CoV2 membrane (M) protein, and a receptor-binding domain of SARS-CoV2 spike protein (S_{RBD}).

20. The fusion protein of claim 4, further comprising a CD28-activating peptide located between the CD40-binding domain and the furin and/or cathepsin L cleavage site, the CD28-activating peptide consisting of 28-53 amino acid residues in length and comprising an amino acid sequence selected from the group consisting of SEQ ID NO: 35, 36 and 37.
